(19) United States
(12) Plant Patent Application Publication
(10) Pub. No.: US 2003/0093847 P1
(43) Pub. Date: May 15, 2003

(54) Begonia Plant Named 'Kristy Franje'

(75) Inventor: Jan Man, Amstelveen (NL)

Correspondence Address: Russell D. Orkin, 700 Koppers Building, 436 Seventh Avenue, Pittsburgh, PA, 15219-1818, US

(73) Assignee: Begonia Breeders Association B.V.

(21) Appl. No.: 10/007,711

(22) Filed: Nov. 13, 2001

(51) Int. Cl. A01H 5/00

(52) U.S. Cl. PLT/348

(57) ABSTRACT

A new and distinct begonia plant with orange colored double flowers and dark green foliage that is resistant to mildew.

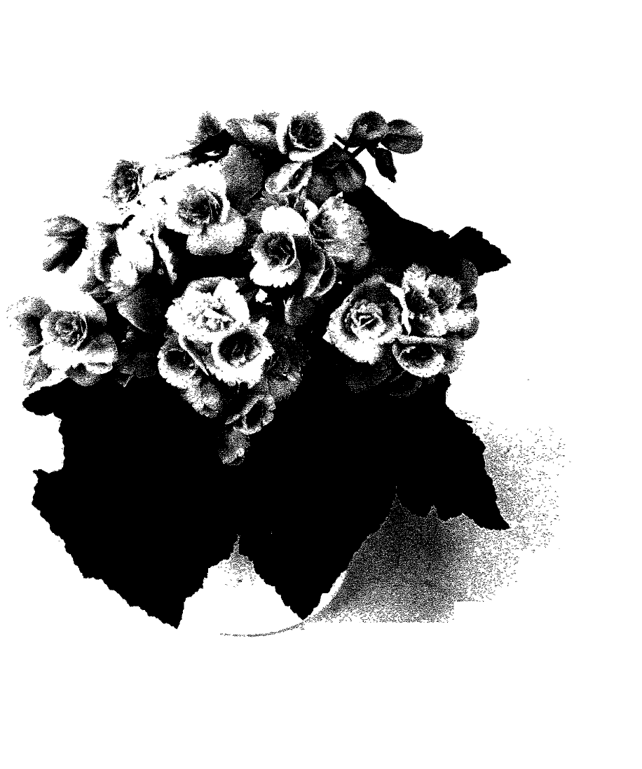

### BACKGROUND OF THE INVENTION

The present invention comprises a new and distinct cultivar of Elatior Begonia, botanically known as Begonia xhiemalis and known by the varietal name ‘Kristy Franje’. The new cultivar was discovered in a controlled environment in Amstelveen, Netherlands, in 1998 as a mutation of ‘Netja Franje’ (unpatented). The new cultivar was observed as a single plant in a group of plants of the parent cultivar. The selection of this plant was based on its unique flower color. The new cultivar differs from its parent as follows:

| | Netje Franje | Kristy Franje |
|---|---|---|
| Outer tepal, middle | Red Group 48D | Red Group 49C |
| upper side | | |
| Outer tepal, margin | Red Group 39D | Orange Group 29D |
| upper side | | |
| Outer tepal, middle | Orange-White Group 159D | Red Group 36D |
| lower side | | |
| Inner tepal, margin | Red Group 52C | Red Group 49C |
| upper side | | |
| Inner tepal, margin | Red Group 52D | Red Group 48D |
| lower side | | |

The new variety was first asexually reproduced by cuttings in February of 1998. The new variety has been trial and field tested and has been found to retain its distinctive characteristics and remain true to type through successive propagations.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions by cuttings in a controlled greenhouse environment. Color references are primarily to the R.H.S. Colour Chart of The Royal Horticultural Society of London.

### PLANT

Classification:

Botanical.—Begonia xhiemalis.

Commercial.—Elatior Begonia.

Form: Upright.

Height: At least about 40 cm tall from top of media surface, 30 cm wide in a 15 cm container 10 weeks after planting, with pinching.

Growth: Moderate growth rate.

Time to initiate roots: About 14 days from a terminal cutting when grown in a glass greenhouse.

Time to develop roots: About 18 days at 22° C.

Rooting habit: Fine roots.

Lateral branches:

Appearance/aspect.—Cylindrical; upright to somewhat outwardly angled.

Strength.—Very good.

Length.—About 19 cm.

Diameter.—About 1 cm.

Internode length.—About 3.8 cm.

Texture.—Mostly smooth with sparse small white hairs; fleshy.

Leaves:

Arrangement.—Alternate.

Shape.—Rounded with acute apex and cordate base with overlap.

Shape.—14 cm wide; 15 cm long.

Quantity: About 7 per lateral branch.

Stipules.—2 per leaf sized 5 mm×5 mm.

Margin.—Bi-crenate.

Texture.—Smooth; shiny and leathery.

Color.—Upper surface: Green Group 136A. Lower surface: Green Group 137A.

Venation.—Palmate, light green.

Petiole.—4.5 cm long; 5 mm in diameter.

### FLOWERS

Habit: Continuous flowering.

Size: 4 cm in diameter; 1 cm deep.

Tepals:

Arrangement: Double.—14-20 tepals per flower.

Size of largest tepal.—2 cm long; 3 cm wide.

Shape.—Oval.

Margin.—Crenate.

Color.—Upper surface: Outer petals: Margin upper side: Orange Group 29D. Middle upper side: Red Group 49C. Inner petals: Margin upper side: Red Group 49C. Middle upper side: Red Group 48D. Lower surface: Red Group 36D to Red Group 49C.

Flower buds: About 1.8 cm long (in autumn); about 1.3 cm wide.

Peduncles:

Angle.—Mostly erect.

Strength.—Strong but flexible.

Length.—About 3.5-4 cm.

Color.—Yellow-Green Group 146B.

Pedicels:

Angle.—About 30 to 45° from vertical.

Strength.—Strong but flexible.

Length.—About 1 cm.

Color.—Yellow-Green Group 146B.

Bracts:

Arrangement.—Two; opposite.

Shape.—Very broadly cordate; acute apex.

Margin.—Entire.

Color.—Light green.

Postproduction longevity:

Flowers.—3-4 weeks.

Plant.—7-8 weeks.

### REPRODUCTIVE ORGANS

Stamens: None observed.

Pistils: None observed.

Disease resistance: Mildew tolerant.

Seed production: Seed production has not been observed as reproductive organs are not formed.

I claim:

1. A new and distinct variety of begonia plant substantially as shown and described.
